(19) United States
(12) Patent Application Publication
(10) Pub. No.: US 2006/0281191 A1
(43) Pub. Date: Dec. 14, 2006

(54) Method for monitoring organic deposits in papermaking

(76) Inventors: Prasad Duggirala, Naperville, IL (US); Sergey Shevchenko, Aurora, IL (US)

Correspondence Address: NALCO COMPANY, 1601 W. DIEHL ROAD, NAPERVILLE, IL, 60563-1198, US

(21) Appl. No.: 11/148,639

(22) Filed: Jun. 9, 2005

(51) Int. Cl. G01N 33/00

(52) U.S. Cl. 436/178

(57) ABSTRACT

A method for monitoring the deposition of organic deposits from a liquid or slurry in a papermaking process is disclosed. The method involves measuring the rate of deposition of organic deposits from the liquid or slurry of a papermaking process on to a quartz crystal microbalance having a top side in contact with the liquid or slurry and a second, bottom side isolated from the liquid or slurry. Also disclosed is a method for measuring the effectiveness of inhibitors that decrease the deposition of organic deposits in a papermaking process. The method involves monitoring the deposition of organic deposits of a liquid or slurry from a papermaking process or from a liquid or slurry that simulates a liquid or slurry found in a papermaking process. Either method comprises measuring the rate of deposition of organic deposits from the liquid or slurry on to a quartz crystal microbalance having a top side in contact with the liquid or slurry and a second, bottom side isolated from the liquid or slurry; adding an inhibitor that decreases the deposition of organic deposits to the liquid or slurry; and re-measuring the rate of deposition of organic deposits from the liquid or slurry on to the quartz crystal microbalance.

### FIELD OF THE INVENTION

This invention is in the field of papermaking. Specifically, this invention is in the field of monitoring organic deposit formation in a papermaking process.

### BACKGROUND OF THE INVENTION

Formation of deposits of organic resinous substances (wood extractives and related natural materials in virgin raw material, stickies and similar man-made components in recycled material) is a common problem in papermaking. For paper grades, these extractives, when liberated during processing of wood or recycled paper products, can become both undesirable components of papermaking furnishes and troublesome deposits on all mill equipment.

The nature of the organic deposits differs from process to process and from mill to mill. Most often, they are mixtures of organic insoluble salts, unsaponifiable organics, wood fibers and/or poorly soluble polymeric paper additives. Thereby, their deposition during the production process is a quite complex matter due to these many possible potential causes.

An express method for organic deposit monitoring and prediction of the activities of deposit control programs is of great value to the industry. Currently, there is no such method in the market.

### SUMMARY OF THE INVENTION

The present invention provides for a method for monitoring the deposition of organic deposits from a liquid or slurry in a papermaking process comprising measuring the rate of deposition of organic deposits from the liquid or slurry on to a quartz crystal microbalance having a top side in contact with the liquid or slurry and second bottom side isolated from the liquid or slurry.

The present invention also provides for a method for measuring the effectiveness of inhibitors that decrease the deposition of organic deposits in a papermaking process comprising monitoring the deposition of organic deposits from a liquid or slurry in a papermaking process comprising measuring the rate of deposition of organic deposits from the liquid or slurry on to a quartz crystal microbalance having a top side in contact with the liquid or slurry and second bottom side isolated from the liquid or slurry; adding an inhibitor that decreases the deposition of organic deposits to the liquid or slurry; and re-measuring the rate of deposition of organic deposits from the liquid or slurry on to the quartz crystal microbalance.

The present invention also provides for a method for measuring the effectiveness of inhibitors that decrease the deposition of organic deposits in a papermaking process comprising: monitoring the deposition of organic deposits from a liquid or slurry that simulate a liquid or slurry found in a papermaking process comprising measuring the rate of deposition of organic deposits from the liquid or slurry on to a quartz crystal microbalance having a top side in contact with the liquid or slurry and a second, bottom side isolated from the liquid or slurry; adding an inhibitor that decreases the deposition of organic deposits to the liquid or slurry; and re-measuring the rate of deposition of organic deposits from the liquid or slurry on to the quartz crystal microbalance.

### BRIEF DESCRIPTION OF THE DRAWINGS

FIG. 1. Formation of organic deposits in the post-oxygen brownstock washer line: mass accumulation.

FIG. 2. Formation of organic deposits in the post-oxygen brownstock washer line: damping voltage.

FIG. 3. Deposition of wood resins and glued fines in the paper machine (white water line).

FIG. 4. Deposition of wood resins and glued fines in the paper machine (white water line): mass accumulation.

FIG. 5. Deposition of wood resins and glued fines in the paper machine (white water line): damping voltage.

FIG. 6. Stickies monitoring in headbox furnish repulped at 60 C (benchtop experiment): mass accumulation.

FIG. 7. Stickies monitoring in headbox furnish repulped at 60 C (benchtop experiment): damping voltage.

FIG. 8. Stickies monitoring in headbox furnish repulped at 60 C (benchtop experiment): temperature.

FIG. 9. Mixed organic/inorganic deposition in D100 filtrate discharge lines of a bleach plant.

FIG. 10. Mixed organic/inorganic deposition in D1 filtrate discharge lines of a bleach plant.

FIG. 11. Mixed aluminum-calcium salt of a polymeric organic acid (a scale inhibitor overdose, diagnostics in deposit control program applications) in a white water line in the broke repulper: mass accumulation.

FIG. 12. Mixed aluminum-calcium salt of a polymeric organic acid (a scale inhibitor overdose, diagnostics in deposit control program applications) in a white water line in the broke repulper: damping voltage.

### DETAILED DESCRIPTION OF THE INVENTION

“QCM” means quartz crystal microbalance.

“IDM” means independent deposition monitor. The instrument is available from Nalco Company, Naperville, Ill. It is a portable instrument that records actual deposition and, from the application standpoint, differs from conventional coupons by its high sensitivity and ability to continuously follow deposition and assess the nature of the deposit. Data are collected continuously at intervals ranging from minutes to hours and then downloaded from the IDM to a personal computer. All plumbing is generally accomplished using stainless steel tubing with compression fittings. This includes the system's sample inlet and outlet. The flow rate in a continuous operation (the probe connected to a process line through a slipstream arrangement) is normally 2-4 gallons per minutes. The instrument also allows data collection from a batch system, where the instrument probe is immersed into the test liquid stirred using a mechanical or magnetic stirrer.

The monitoring system is based on the QCM that is the main part of the instrument's probe. Basic physical principles and terminology of the QCM can be found in publications: Martin et al., Measuring liquid properties with smooth- and textured-surface resonators, Proc. IEEE Int. Freq. Control Symp., v. 47, p. 603-608 (1993); Martin et al., Resonator/Oscillator response to liquid loading, Anal. Chem., v. 69 (11), 2050-2054 (1997); Schneider et. al., Quartz Crystal Microbalance (QCM) arrays for solution analysis, Sandia Report SAND97-0029, p. 1-21 (1997). In the QCM, a flat quartz crystal is sandwiched between two electrically conductive surfaces. One surface (top side) is in a continuous contact with the tested medium while the other (bottom side) is isolated from the tested liquid or slurry. The QCM vibrates when the electrical potential is applied (piezoelectric effect). The parameters measured by the instrument probe, oscillator frequency and damping voltage are connected to the amount and physical properties of the deposit on the top (open to the medium) side of the QCM. The vibration frequency is, generally, linearly proportional to the mass of a deposit on the metal surface of the QCM. Measuring the frequency thus provides a means to monitor real-time deposition. The instrument also measures damping voltage. This parameter is dependent on the viscoelastic properties of the deposit thus being indicative of its nature. Damping voltage does not change in case of rigid deposits (any inorganic scale). It increases during the initial stage of accumulation in case of organic deposits. Both oscillator frequency and damping voltage are also affected by the properties of the aqueous phase such as a temperature and viscosity. Therefore, uniform conditions should be maintained through every experiment.

In one embodiment, the papermaking process occurs at location selected from the group consisting of: a pulp mill; a papermaking machine; a tissue making machine; a repulper; water loop; wet-end stock preparation; and deinking stages.

In another embodiment, the organic deposits are selected from the group consisting of: wood; extractives; redeposited lignin; defoamers; surfactants; and stickies. In another embodiment, the surfactants are silicon surfactants.

In another embodiment, the stickies are selected from the group consisting of: sizing chemicals; and adhesives.

In another embodiment, the continuously flowing slurry is a pulp slurry.

In another embodiment, said organic deposits are silicon surfactants and said papermaking process is a tissue repulping process.

In another embodiment, the top side of the quartz crystal microbalance is made of one or more conductive materials selected from the group consisting of: platinum; titanium; silver; gold; lead; cadmium; diamond-like thin film electrodes with or without implanted ions; suicides of titanium, niobium and tantalum; lead-selenium alloys; mercury amalgams; and silicon.

In another embodiment, the top side of the quartz crystal microbalance is coated with any one or more conductive or unconductive materials selected from the group consisting of: polymeric films; monolayers; polylayers; surfactants; polyelectrolites; thiols; silica; aromatic sorbates; self-assembled monolayers; and molecular solids.

The following examples not meant to limit the invention unless otherwise stated in the claims appended hereto.

### EXPERIMENTS

### Example 1

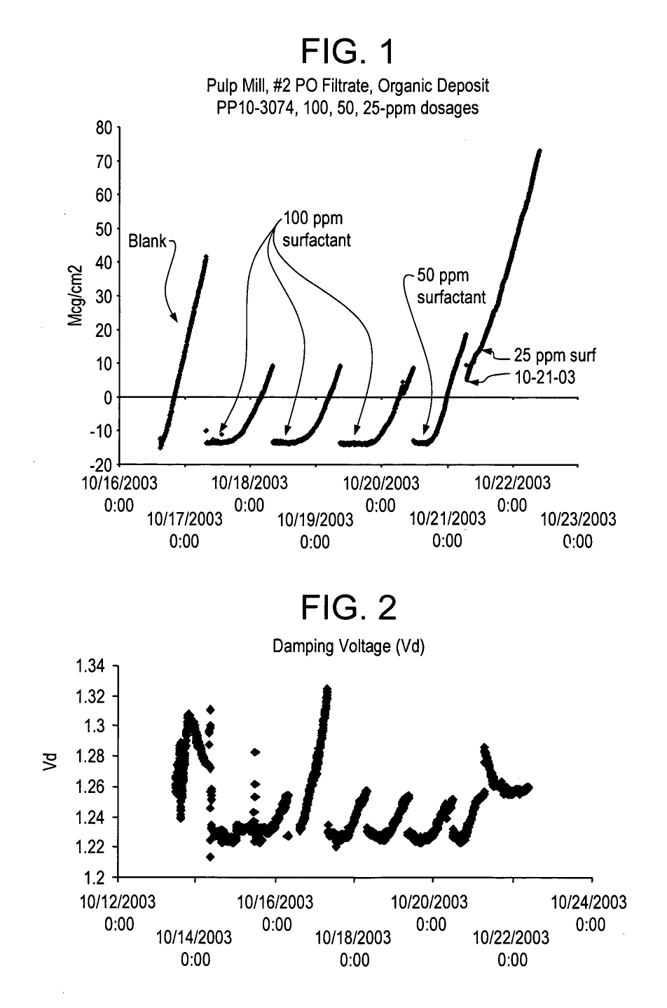

The IDM instrument was directly connected (a slipstream connection) to a filtrate line to assure a continuous flow of the solution. The deposition was directly recorded and the data is embodied in FIG. 1 and FIG. 2. Formation of “light” organic deposits in a post-oxygen brownstock washer line was monitored on-line with the IDM. Steady mass accumulation was observed accompanied by characteristic changes in damping voltage (an initial increase followed by flattening). In several experiments, the addition of Nalco chemical PP10-3095 led to deposit removal followed by complete suppression of deposition (100-50 ppm) or slowing the deposition down (25 ppm).

### Example 2

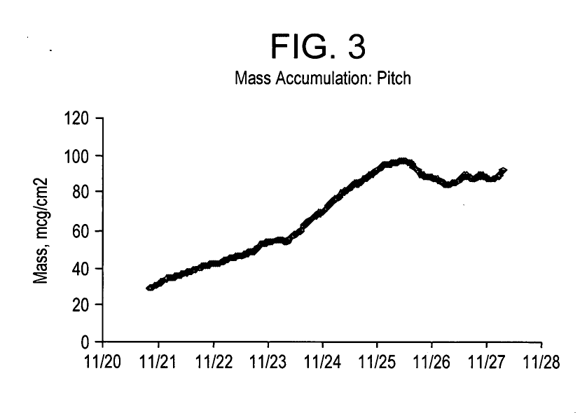

The IDM instrument was directly connected (a slipstream arrangement) to the white water line in the paper machine (0.3-0.5% pulp fines). The deposition of wood resins and glued fines was directly recorded and the data is embodied in FIG. 3. The deposition stopped when Nalco chemical PP10-3095 was applied at 100 ppm (note that the chemical did not remove the material from the surface of the QCM).

### Example 3

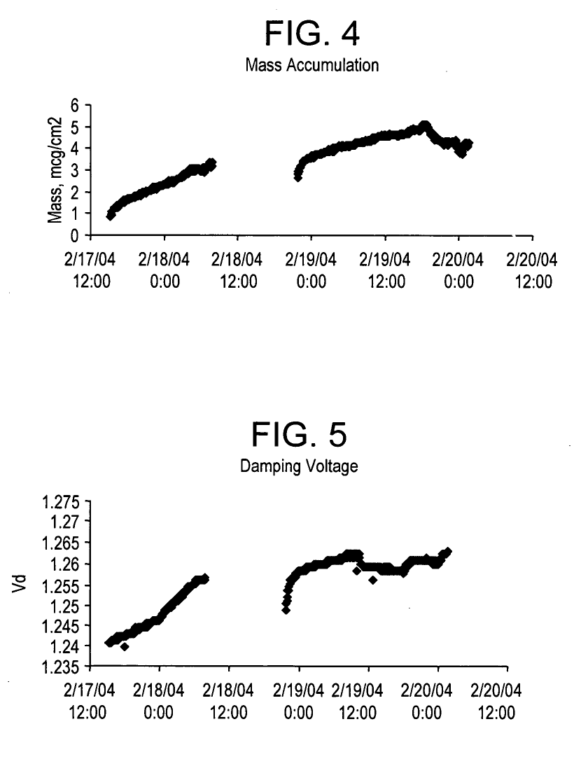

The IDM instrument was directly connected (a slipstream arrangement) to the white water line in the paper machine (0.3-0.5% pulp fines). The deposition of wood resins and glued fines was recorded and the data is embodied in FIG. 4 and FIG. 5. The deposition stopped when Nalco chemical PP10-3095 was applied at 50 ppm and 100 ppm (the chemical did not remove pitch from the surface of the QCM).

### Example 4

Silicon oil surfactants from facial tissue repulping process (3% pulp, beaker, 400 rpm, room temperature). In this benchtop application, linear accumulation of the organic deposit was observed, at a rate dependent of presence of deposit control agents in the system.

### Example 5

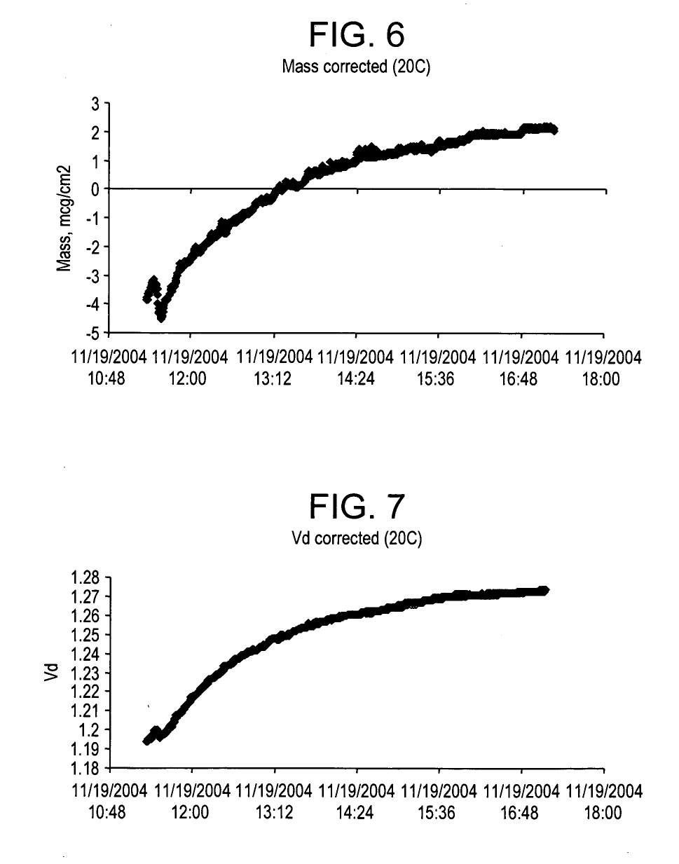

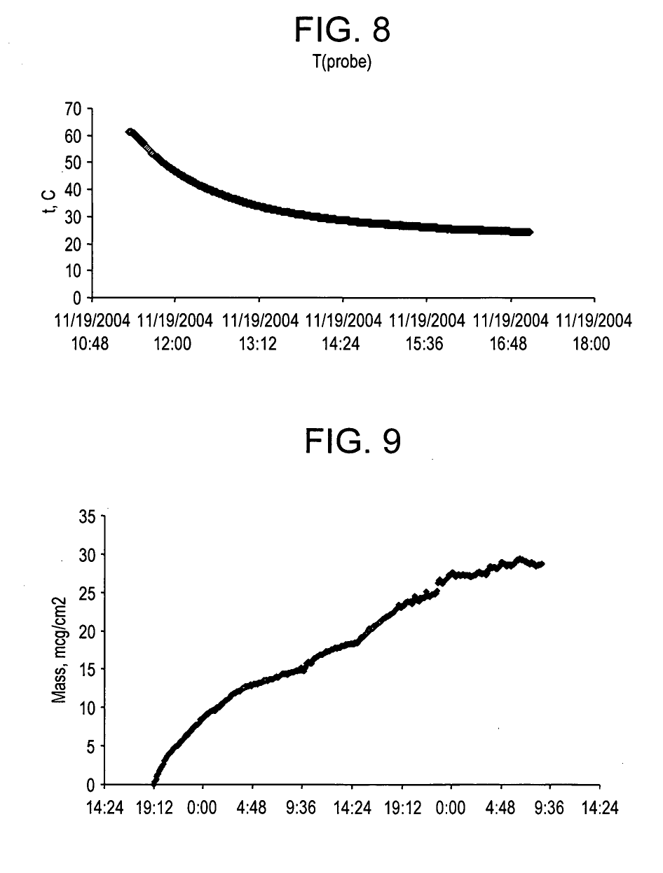

Stickies monitoring. A sample of headbox furnish (100% recycled OCC box) was repulped at 60 C. The slurry was transferred in a 1-L beaker with a magnetic stirrer. The IDM probe was placed vertically on a stand and the data is embodied in FIGS. 6-8. The slurry was stirred at a constant rate 400 rpm at room temperature and allowed to cool down. The data are corrected to 20 C using the temperature-frequency linear correlation formula obtained for the IDM instrument in a separate experiment. Mass accumulation and damping voltage curves could be unambiguously ascribed to an organic material that deposits at a noticeable rate while the solution is still warm, later deposition slowed down.

### Example 6

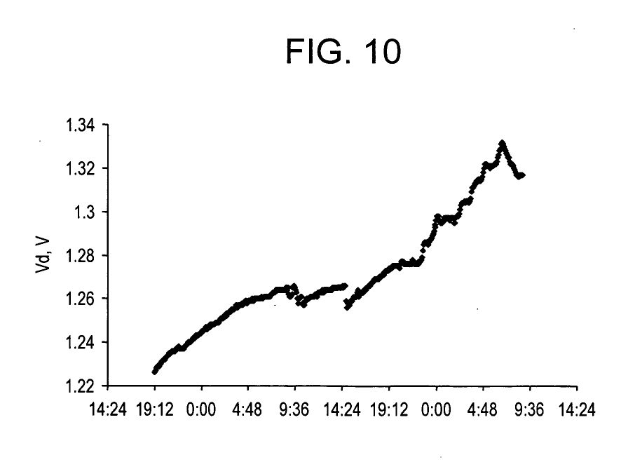

Mixed organic/inorganic deposits. This gives an example of using the technique as both a monitoring and diagnostic tool. In a paper mill, the IDM was installed, consecutively, in filtrate discharge lines (pH 3.5-3.8, 60-66° C.) where mixed barium sulfate/calcium oxalate scale was thought to be depositing. In both cases, the instrument recorded deposition that could not be ascribed entirely to an inorganic scale due to noticeable changes in damping voltage. (See FIGS. 9-10). Indeed, microphotographs of the deposit also indicated that the scale is mixed, predominantly containing an organic component (likely, trapped fibers and possibly viscous organic).

### Example 7

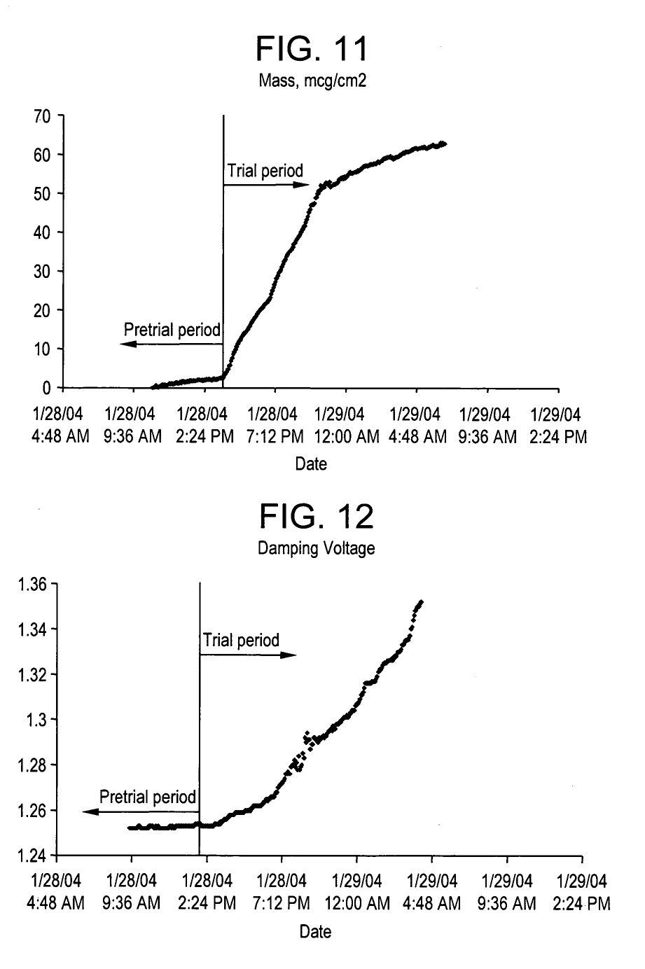

Mixed aluminum-calcium salt of a polymeric organic acid (a scale inhibitor overdose, diagnostics in deposit control program applications). The IDM instrument was directly connected (a slipstream arrangement) to the white water line in the broke repulper (0.3-0.5% pulp fines). The deposition initially was inorganic. The solution contained very high concentrations of metal ions, especially aluminum and calcium. Application of an excess of a scale control agent into the IDM line via peristaltic pump that was a polymeric organic acid in its nature resulted in a surge of deposition. (See FIGS. 11-12). The instrument allowed to immediately ascribe this phenomenon to an organic material that could only be a mixed aluminum-calcium salt of a polymeric organic acid formed due to scale inhibitor overdose.

What is claimed is:

1. A method for monitoring the deposition of organic deposits from a liquid or slurry in a papermaking process comprising measuring the rate of deposition of organic deposits from the liquid or slurry on to a quartz crystal microbalance having a top side in contact with the liquid or slurry and a second, bottom side isolated from the liquid or slurry.

2. The method of claim 1 wherein the top side of the quartz crystal microbalance is made of one or more conductive materials selected from the group consisting of: platinum; titanium; silver; gold; lead; cadmium; diamond-like thin film electrodes with or without implanted ions; silicides of titanium, niobium and tantalum; lead-selenium alloys; mercury amalgams; and silicon.

3. The method of claim 1 wherein said papermaking process occurs at location selected from the group consisting of: a pulp mill; a papermaking machine; a tissue making machine; a repulper; water loop; wet-end stock preparation; and deinking stages.

4. The method of claim 1 wherein said organic deposits are selected from the group consisting of: wood; extractives; redeposited lignin; defoamers; surfactants; and stickies.

5. The method of claim 4 wherein said stickies are selected from the group consisting of: sizing chemicals; and adhesives.

6. The method of claim 1 wherein said slurry is a pulp slurry.

7. A method for measuring the effectiveness of inhibitors that decrease the deposition of organic deposits in a papermaking process comprising:
a. monitoring the deposition of organic deposits from a liquid or slurry in a papermaking process comprising measuring the rate of deposition of organic deposits from the liquid or slurry on to a quartz crystal microbalance having a top side in contact with the liquid or slurry and a second, bottom side isolated from the liquid or slurry;
b. adding an inhibitor that decreases the deposition of organic deposits to the liquid or slurry; and
c. re-measuring the rate of deposition of organic deposits from the liquid or slurry on to the quartz crystal microbalance.

8. The method of claim 7 wherein said papermaking process occurs at location selected from the group consisting of: a pulp mill; a papermaking machine; a tissue making machine; a repulper; water loop; wet-end stock preparation; and deinking stages.

9. A method for measuring the effectiveness of inhibitors that decrease the deposition of organic deposits in a papermaking process comprising:
a. monitoring the deposition of organic deposits from a liquid or slurry that simulate a liquid or slurry found in a papermaking process comprising measuring the rate of deposition of organic deposits from the liquid or slurry on to a quartz crystal microbalance having a top side in contact with the liquid or slurry and a second, bottom side isolated from the liquid or slurry;
b. adding an inhibitor that decreases the deposition of organic deposits to the liquid or slurry; and
c. re-measuring the rate of deposition of organic deposits from the liquid or slurry on to the quartz crystal microbalance.

10. The method of claim 4, wherein said surfactants are silicon surfactants.

11. The method of claim 1, wherein said organic deposits are silicon surfactants and said papermaking process is a tissue repulping process.

12. The method of claim 1 wherein the top side of the quartz crystal microbalance is coated with any one or more conductive or unconductive materials selected from the group consisting of: polymeric films; monolayers; polylayers; surfactants; polyelectrolites; thiols; silica; aromatic sorbates; self-assembled monolayers; and molecular solids.
